(19) United States
(12) Patent Application Publication
(10) Pub. No.: US 2025/0090432 A1
(43) Pub. Date: Mar. 20, 2025

(54) Oral Care Compositions

(71) Applicant: Colgate-Palmolive Company, New York, NY (US)

(72) Inventors: Shaoyi ZHANG, South Bound Brook, NJ (US); Bayardo GARCIA GODOY, Princeton, NJ (US); Divino RAJAH, Morristown, NJ (US); Darrick CARLONE, New Vernon, NJ (US); Connor MCCORMICK, Stewartsville, NJ (US); Suman CHOPRA, Monroe, NJ (US)

(73) Assignee: Colgate-Palmolive Company, New York, NY (US)

(21) Appl. No.: 18/884,323

(22) Filed: Sep. 13, 2024

Related U.S. Application Data
(60) Provisional application No. 63/582,593, filed on Sep. 14, 2023.

(51) Int. Cl. A61K 8/22; A61K 8/19; A61K 8/21; A61K 8/81; A61Q 11/00

(52) U.S. Cl. CPC A61K 8/22; A61K 8/19; A61K 8/21; A61K 8/8176; A61Q 11/00

(57) ABSTRACT

Oral care compositions are disclosed herein. In accordance with an aspect, provided is an oral care composition having a whitening agent; potassium nitrate, wherein about 80% or more of the total amount of potassium nitrate has a particle size of about 150 μm or less; water in an amount of about 8 wt. % or less or about 3 wt. % or less, wherein all weight percentages are based on the total weight of the oral care composition.

### CROSS-REFERENCE TO RELATED APPLICATIONS

This application claims the benefit of priority from U.S. Patent Application No. 63/582,593, filed Sep. 14, 2023, titled Oral Care Compositions, the content of which is hereby incorporated herein in its entirety for all purposes.

### BACKGROUND

Many individuals desire a “bright” smile and white teeth, and consider dull and stained teeth cosmetically unattractive. Thus, there is a desire for whiter teeth and one means to achieve whiter teeth is the use of tooth whitening products.

Teeth can become discolored by foods, drinks and tobacco use. Dental stains can be classified as either extrinsic, which occur on the outer surface of teeth, or intrinsic, which occur below the surface of enamel. Most abrasive containing toothpaste remove extrinsic stains. Peroxides can bleach both extrinsic and intrinsic stains and so provides fast and superior whitening efficacy. The peroxide can bleach the teeth, remove stains, and kill cariogenic bacteria.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions. In accordance with an aspect, provided is an oral care composition having a whitening agent; potassium nitrate, wherein about 80% or more of the total amount of potassium nitrate has a particle size of about 500 μm or less; water in an amount of about 8 wt. % or less or about 3 wt. % or less, wherein all weight percentages are based on the total weight of the oral care composition.

A list of non-limiting example embodiments is provided below:

In accordance with an embodiment 1, provided is an oral care composition comprising:
a whitening agent;
potassium nitrate, wherein about 80% or more of the total amount of potassium nitrate has a particle size of about 500 μm or less; and
water in an amount of about 8 wt. % or less, wherein all weight percentages are based on the total weight of the oral care composition.

In accordance with an embodiment 2, provided is an oral care composition according to claim 1,
wherein the oral care composition is substantially free, or free, of water.

In accordance with an embodiment 3, provided is an oral care composition according to any foregoing embodiment, wherein about 99% or more of the total amount of potassium nitrate has a particle size of about 2 mm or less.

In accordance with an embodiment 4, provided is an oral care composition according to embodiment 3, wherein about 99% or more of the total amount of potassium nitrate has a particle size of about 1.2 mm or less.

In accordance with an embodiment 5, provided is an oral care composition according to any foregoing embodiment, wherein about 80% or more of the total amount of potassium nitrate has a particle size of about 150 μm or less.

In accordance with an embodiment 6, provided is an oral care composition according to any foregoing embodiment, wherein the whitening agent is selected from hydrogen peroxide, cross-linked polyvinylpyrrolidone complexed with hydrogen peroxide, vinylpyrrolidone copolymer complexed with hydrogen peroxide, a silica hydrogen peroxide complex, and a combination of two or more thereof.

In accordance with an embodiment 7, provided is an oral care composition according to any foregoing embodiment, wherein the whitening agent comprises polyvinylpyrrolidone complexed with hydrogen peroxide.

In accordance with an embodiment 8, provided is an oral care composition according to embodiment 7, wherein the whitening agent further comprises hydrogen peroxide.

In accordance with an embodiment 9, provided is an oral care composition according to any foregoing embodiment, wherein the whitening agent is present in an amount from about 1 to about 40 wt. %.

In accordance with an embodiment 10, provided is an oral care composition according to any foregoing embodiment, wherein the total amount of hydrogen peroxide, including both complexed and non-complexed hydrogen peroxide, present in the oral care composition is from about 1 to about 8 wt. %.

In accordance with an embodiment 11, provided is an oral care composition according to any foregoing embodiment further comprising a polyol, optionally, in amount for from about 15 to about 72 wt. %.

In accordance with an embodiment 12, provided is an oral care composition according to embodiment 11, wherein the polyol is selected from propylene glycol, a block copolymer of ethylene oxide and propylene oxide, a polyethylene glycol/polypropylene glycol copolymer, glycerin, sorbitol, and a combination of two or more thereof.

In accordance with an embodiment 13, provided is an oral care composition according to any foregoing embodiment further comprising a fatty amphiphilic.

In accordance with an embodiment 14, provided is an oral care composition according to embodiment 13, wherein the fatty amphiphilic is selected from fatty alcohols, alkoxylated fatty alcohols, fatty phenols, alkoxylated fatty phenols, fatty amides, alkyoxylated fatty amides, fatty amines, fatty alkylamidoalkylamines, fatty alkyoxylated amines, fatty carbamates, fatty amine oxides, fatty acids, alkoxylated fatty acids, fatty diesters, fatty sorbitan esters, fatty sugar esters, methyl glucoside esters, fatty glycol esters, mono, di- and tri-glycerides, polyglycerine fatty esters, alkyl glyceryl ethers, propylene glycol fatty acid esters, cholesterol, ceramides, fatty silicone waxes, fatty glucose amides, phospholipids, and a combination of two or more thereof.

In accordance with an embodiment 15, provided is an oral care composition according to any foregoing embodiment further comprising an abrasive, optionally, in an amount from about 1 to about 30 wt. %.

In accordance with an embodiment 16, provided is an oral care composition according to embodiment 15, wherein the abrasive is selected from alumina, insoluble phosphates, calcium carbonate, resinous abrasives, silica, and a combination of two or more thereof.

In accordance with an embodiment 17, provided is an oral care composition according to any foregoing embodiment further comprising an anti-calculus agent in an amount from about 0.5 to about 15 wt. %.

In accordance with an embodiment 18, provided is an oral care composition according to embodiment 17, wherein the anti-calculus agent is selected from selected from tetrasodium pyrophosphate, ammonium tripolyphosphate, hexametaphosphate salt, and a combination thereof.

In accordance with an embodiment 19, provided is an oral care composition according to any foregoing embodiment further comprising a sweetener, optionally, in an amount from about 0.1 to about 7 wt. %.

In accordance with an embodiment 20, provided is an oral care composition according to embodiment 19, wherein the sweetener is selected from sucrose, lactose, maltose, sorbitol, xylitol, sodium cyclamate, perillartine, L-aspartyl-L-phenylalanine methyl ester, saccharine, and a combination of two or more thereof.

In accordance with an embodiment 21, provided is an oral care composition according to any foregoing embodiment further comprising a fluoride source, optionally, in an amount from about 0.1 to about 2 wt. %.

In accordance with an embodiment 22, provided is an oral care composition according to embodiment 21, wherein the fluoride source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

In accordance with an embodiment 23, provided is an oral care composition according to any foregoing embodiment further comprising an anionic surfactant, optionally, in an amount from about 0.5 to about 10 wt. %.

In accordance with an embodiment 24, provided is an oral care composition according to embodiment 23, wherein the anionic surfactant is selected from ammonium lauryl sulfate, ammonium lauryl ether sulfate, sodium dodecyl sulfate, sodium coco-sulfate, ammonium coco-sulfate, and a combination of two or more thereof. In some preferred embodiments, the oral care composition comprises sodium lauryl sulfate, sodium lauryl ether sulfate, or a combination of thereof.

In accordance with an embodiment 25, provided is an oral care composition comprising:
a whitening agent;
potassium nitrate, wherein about 80% or more of the total amount of potassium nitrate has a particle size of about 150 μm or less;
from about 15 to about 72 wt. % of a polyol, wherein the polyol consists of a glycol, a polypropylene glycol (PPG), a polyethylene glycol (PEG), a block copolymer of PPG and PEG a block copolymer of ethylene oxide and propylene oxide, glycerin, mannitol, maltitol, xylitol, inositol, sorbitol, a block copolymer of polyoxypropylene and polyoxyethylene and a combination of two or more thereof; and about 3 wt. % or less of water, wherein all weight percentages are based on the total weight of the oral care composition.

In accordance with an embodiment 26, provided is an oral care composition according to any foregoing embodiment, wherein the oral care composition provides a reduction in tactile hypersensitivity of about 55% or more after 4 weeks of using the oral care composition at least twice a day.

In accordance with an embodiment 27, provided is an oral care composition according to any foregoing embodiment, wherein the oral care composition provides a reduction in tactile hypersensitivity of about 84% or more after 8 weeks of using the oral care composition at least twice a day.

In accordance with an embodiment 28, provided is an oral care composition according to embodiment 26 or embodiment 27, wherein the reduction in tactile hypersensitivity is provided using the oral care composition at least once in the morning at least once in the evening.

In accordance with an embodiment 29, provided is an oral care composition according to any foregoing embodiment, wherein the oral care composition provides a reduction in air blast hypersensitivity of about 28% or more after 4 weeks of using the oral care composition at least twice a day.

In accordance with an embodiment 30, provided is an oral care composition according to any foregoing embodiment, wherein the oral care composition provides a reduction in air blast hypersensitivity of about 41% or more after 8 weeks of using the oral care composition at least twice a day.

In accordance with an embodiment 31, provided is an oral care composition according to embodiment 29 or embodiment 30, wherein the reduction in air blast hypersensitivity is provided using the oral care composition at least once in the morning at least once in the evening.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both a polyol and a sweetener. If a particular oral care composition includes both a polyol and a sweetener, xylitol will serve only as either a polyol or a sweetener—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions. In accordance with an aspect, provided is an oral care composition having a whitening agent; potassium nitrate, wherein about 80% or more of the total amount of potassium nitrate has a particle size of about 500 μm or less; water in an amount of about 8 wt. % or less or 3 wt. % or less, wherein all weight percentages are based on the total weight of the oral care composition.

Preferably, the oral care compositions achieve an improvement in tactile hypersensitivity (e.g., a reduction in tactile sensitivity) of about 55% or more after 4 weeks of using (e.g., brushing with) the oral care composition at least twice a day (e.g., at least once in the morning and at least once in the evening). In certain embodiments, the oral care composition provides an improvement in tactile hypersensitivity of about 62% or more, about 67% or more, about 72% or more, about 77% or more, or about 82% or more, after 4 weeks of using the oral care composition at least twice a day (e.g., at least once in the morning and at least once in the evening). In some embodiments, the foregoing percentage improvements for the oral care composition is relative to a comparative composition having a formulation having a hole instead of the potassium nitrate.

Additionally or alternatively, the oral care compositions achieve an improvement in tactile hypersensitivity (e.g., a reduction in tactile hypersensitivity) of about 84% or more after 8 weeks of using (e.g., brushing with) the oral care composition at least twice a day (e.g., at least once in the morning and at least once in the evening). In some preferred embodiments, the oral care compositions provide an improvement in tactile hypersensitivity of about 91% or more, about 96% or more, about 100% or more, about 105%, or about 110% or more, about 115% or more, or about 120% or more after 8 weeks of using the oral care composition at least twice a day (e.g., at least once in the morning and at least once in the evening).

The oral care compositions may preferably achieve an improvement in air blast hypersensitivity (e.g., a reduction in air blast hypersensitivity) of about 28% or more after 4 weeks of using (e.g., brushing with) the oral care composition at least twice a day (e.g., at least once in the morning and at least once in the evening). In certain embodiments, the oral care compositions provide an improvement in air blast hypersensitivity of about 29% or more, about 32% or more, about 35% or more, about 38% or more, or about 42% or more, after 4 weeks of using the oral care composition at least twice a day (e.g., at least once in the morning and at least once in the evening). In some embodiments, the foregoing percentage improvements for the oral care composition is relative to a comparative composition having a formulation that is same except for having a hole instead of the potassium nitrate.

The oral care compositions may, additional or alternatively, achieve an improvement in air blast hypersensitivity (e.g., a reduction in air blast sensitivity) of about 41% or more after 8 weeks of using (e.g., brushing with) the oral care composition at least twice a day (e.g., at least once in the morning and at least once in the evening). In certain embodiments, the oral care composition provides an improvement in air blast hypersensitivity of about 45% or more, about 49% or more, about 53% or more, about 57% or more, or about 61% or more, after 8 weeks of using the oral care composition at least twice a day (e.g., at least once in the morning and at least once in the evening). In some embodiments, the foregoing percentage improvements for the oral care composition is relative to a comparative composition having a formulation that is the same except for having a hole instead of the potassium nitrate.

Additionally or alternatively, the oral care compositions typically have less than about 8 wt. % of water. In some embodiments, the amount of added water and/or the total amount of water present in the oral care composition is less than about 7 wt. %, less than about 6 wt. %, less than about 5 wt. %, less than about 4 wt. %, less than about 3 wt. %, less than about 2 wt. %, less than about 1 wt. %, less than about 0.5 wt. %, less than about 0.1 wt. %, based on the total weight of the oral care composition. The oral care composition may be substantially free of or free of added water. In further instances, the oral care composition is substantially free of or free of water. In further instances, the oral care composition may have about 0 wt. % or 0 wt. % of added water, based on the total weight of the oral care composition. In certain instances, the total amount of water in the oral care composition is about 0 wt. % or 0 wt. %, based on the total weight of the oral care composition. As noted above, the amount of added water may be the amount of non-incidental amount of an ingredient or compound that is added into the oral care composition by itself.

Without being limited to any theory, the inventors unexpectedly discovered that certain oral care compositions may be formulated with a reduced amount, being substantially free of, or free of water while achieving one or more of the improvements in hypersensitivity disclosed herein, e.g., as assessed by air blast hypersensitivity and/or tactile hypersensitivity. The inventors discovered that certain oral care compositions having particular ingredients and components in certain amounts can achieve a reduction in dentin hypersensitivity while employing a peroxide and potassium nitrate, which was conventionally believed to not disassociate in anhydrous oral care compositions.

The oral care compositions may be in the form of a dentifrice, a toothpaste, a gel, and/or the like. Suitable components, such as those listed below, may be included or excluded from the formulations for the oral care compositions disclosed herein depending on the specific combination of other ingredients and/or the form of the oral care compositions. Additionally or alternatively, the oral care compositions may in some embodiments have a single phase, which contains that components and/or ingredients of the oral care composition. In other embodiments, the oral care composition may include two or more phases, such as two, three, four, or five phases.

The oral care compositions typically include one or more whitening agent(s), preferably in an effective amount. In some embodiments, the amount of whitening agent(s) in the oral care composition is from about 1 to about 40 wt. %, based on the total weight of the oral care composition. For example, the whitening agent(s) may be present in an amount from about 1 to about 35 wt. %, about 1 to about 24 wt. %, about 1 to about 20 wt. %, about 1 to about 15 wt. %, about 1 to about 12 wt. %, about 1 to about 9 wt. %, about 1 to about 6 wt. %, about 1 to about 4 wt. %; from about 3 to about 40 wt. %, about 3 to about 30 wt. %, about 3 to about 24 wt. %, about 3 to about 20 wt. %, about 3 to about 15 wt. %, about 3 to about 12 wt. %, about 3 to about 9 wt. %, about 3 to about 6 wt. %; from about 5 to about 40 wt. %, about 5 to about 30 wt. %, about 5 to about 24 wt. %, about 5 to about 20 wt. %, about 5 to about 18 wt. %, about 5 to about 15 wt. %, about 5 to about 12 wt. %, about 5 to about 9 wt. %; from about 7 to about 40 wt. %, about 7 to about 35 wt. %, about 7 to about 30 wt. %, about 7 to about 27 wt. %, about 7 to about 24 wt. %, about 7 to about 22 wt. %, about 7 to about 20 wt. %, about 7 to about 18 wt. %, about 7 to about 15 wt. %, about 7 to about 12 wt. %; from about 10 to about 40 wt. %, about 10 to about 35 wt. %, about 10 to about 30 wt. %, about 10 to about 27 wt. %, about 10 to about 24 wt. %, about 10 to about 22 wt. %, about 10 to about 20 wt. %, about 10 to about 18 wt. %, about 10 to about 15 wt. %; from about 14 to about 40 wt. %, about 14 to about 35 wt. %, about 14 to about 30 wt. %, about 14 to about 27 wt. %, about 14 to about 24 wt. %, about 14 to about 22 wt. %, about 14 to about 20 wt. %, about 14 to about 18 wt. %; from about 18 to about 40 wt. %, about 18 to about 35 wt. %, about 18 to about 30 wt. %, about 18 to about 27 wt. %, about 18 to about 24 wt. %, about 18 to about 22 wt. %, about 18 to about 20 wt. %; from about 20 to about 40 wt. %, about 20 to about 35 wt. %, about 20 to about 30 wt. %, about 20 to about 27 wt. %, about 20 to about 24 wt. %; from about 22 to about 40 wt. %, about 22 to about 35 wt. %, about 22 to about 30 wt. %, about 22 to about 27 wt. %, about 22 to about 24 wt. %; from about 24 to about 40 wt. %, about 24 to about 35 wt. %, about 24 to about 30 wt. %, about 24 to about 27 wt. %; from about 27 to about 40 wt. %, about 27 to about 35 wt. %, about 27 to about 30 wt. %; from about 30 to about 40 wt. %, about 30 to about 35 wt. %, about 35 to about 40 wt. %, or any range or subrange thereof, based on the total weight of the oral care composition.

The whitening agent(s) may comprise a peroxide, a metal chlorite, and/or a persulfate. Examples of peroxides include hydrogen peroxide, peroxides of alkali and alkaline earth metals, organic peroxy compounds, peroxy acids, peroxide complexes, salts thereof, or combinations of two or more thereof. Peroxides of alkali and alkaline earth metals include lithium peroxide, potassium peroxide, sodium peroxide, magnesium peroxide, calcium peroxide, barium peroxide, and a combination of two or more thereof. Examples of organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate, or a combination of two or more thereof. Peroxy acids and their salts include organic peroxy acids, such as alkyl peroxy acids, monoperoxyphthalate, and a combination thereof as well as inorganic peroxy acid salts, such as persulfate, dipersulfate, percarbonate, perphosphate, perborate and persilicate salts of alkali and/or alkaline earth metals, such as lithium, potassium, sodium, magnesium, calcium and barium, and mixtures thereof. In various embodiments, the peroxide compound comprises hydrogen peroxide, urea peroxide, sodium percarbonate, or a combination of two or more thereof.

The whitening agent may comprise a peroxide complex. The peroxide complex may comprise a peroxide component and a porous cross-linked polymer. As referred to herein, a “peroxide component” is any oxidizing compound comprising a bivalent oxygen-oxygen group. Peroxide components include peroxides and hydroperoxides disclosed above, such as hydrogen peroxide, peroxides of alkali and alkaline earth metals, organic peroxy compounds, peroxy acids, salts thereof, and mixtures thereof. In various embodiments, the peroxide component comprises hydrogen peroxide, urea peroxide, sodium percarbonate and mixtures thereof. In one embodiment, the peroxide component comprises hydrogen peroxide. In one embodiment, the peroxide component consists essentially or consists of hydrogen peroxide.

The peroxide complex may also be selected from N-vinyl heterocyclic polymers. Preferably, the N-vinyl heterocyclic polymer is crosslinked and adsorbs, absorbs, complexes, or otherwise retains the peroxide component. The chemical and physical characteristics of the particulate hinder the release of the peroxide compound from the polymer particulates, and in some embodiments provides controlled release of the peroxide compound. The peroxide complex may comprise hydrogen peroxide in an amount such that the oral care composition includes from about 1 to about 7 wt. %, about 1 to about 5 wt. %, about 1 to about 4 wt. %, about 1 to about 3 wt. %, about 1 to about 2.5 wt. %, about 1 to about 2 wt. %; from about 1.5 to about 7 wt. %, about 1.5 to about 5 wt. %, about 1.5 to about 4 wt. %, about 1.5 to about 3 wt. %, about 1.5 to about 2.5 wt. %; from about 2.5 to about 7 wt. %, about 2.5 to about 5 wt. %, about 2.5 to about 4 wt. %; from about 3.5 to about 7 wt. %, about 3.5 to about 5 wt. %, about 3.5 to about 4.5 wt. %; from about 4 to about 7 wt. %, about 4 to about 5 wt. %; from about 5 to about 20 wt. %, about 5 to about 15 wt. %, about 5 to about 10 wt. %, about 5 to about 7 wt. %, or any range or subrange thereof, of hydrogen peroxide, based on the total weight of the oral care composition. In one embodiment, the oral care composition includes from about 1 to about 8 wt. %, e.g., from about 1.5 to about 7 wt. %, about 1.5 to about 5 wt. %, about 1.5 to about 4 wt. %, about 1.5 to about 3 wt. %, about 1.5 to about 2.5 wt. %; from about 2.5 to about 7 wt. %, about 2.5 to about 5 wt. %, about 2.5 to about 4 wt. %; from about 3.5 to about 7 wt. %, about 3.5 to about 5 wt. %, about 3.5 to about 4.5 wt. %; from about 4 to about 7 wt. %, about 4 to about 5 wt. %, from about 3 to about 5 wt. %, or about 4 wt. %, based on the total weight of the oral care composition, of hydrogen peroxide, inclusive of complexed and non-complexed hydrogen peroxide.

In some embodiments, however, peroxide(s) may be non-complexed peroxides. The non-complexed peroxides may be chosen from one or more of the peroxides described above. In some embodiments, a non-peroxide whitening agent may be included in the oral care composition. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite, pigments, or dyes.

In at least one embodiment, the whitening agent is selected from hydrogen peroxide, cross-linked polyvinylpyrrolidone complexed with hydrogen peroxide, vinylpyrrolidone copolymer complexed with hydrogen peroxide, silica hydrogen peroxide complex, and a combination of two or more thereof.

The oral care composition typically comprises potassium nitrate, preferably in an effective amount. In some instances, amount of potassium nitrate in the oral care composition is from about 1 to about 7 wt. %, based on the total weight of the oral care composition. For example, the potassium nitrate may be present in an amount from about 1 to about 6 wt. %, about 1 to about 5 wt. %, about 1 to about 4 wt. %, about 1 to about 3 wt. %, about 1 to about 2 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %, about 3 to about 4 wt. %; from about 4 to about 7 wt. %, about 4 to about 6 wt. %, about 4 to about 5 wt. %; from about 5 to about 7 wt. %, about 5 to about 6 wt. %, or about 6 to about 7 wt. %, or any range or subrange thereof, based on the total weight of the oral care composition.

The oral care composition may, in some cases, include a plurality of potassium nitrates. Preferably, about 80% or more of the total amount of potassium nitrate has a particle size of about 500 μm or less. For example, the oral care composition has about 80% or more, e.g., about 85% or more, about 88% or more, about 91% or more, about 95% or more, about 97% or more, or about 99% or more, of the total amount of potassium nitrate having a particle size of about 500 μm or less, e.g., about 350 μm or less, about 250 μm or less, about 200 μm or less, about 150 μm or less.

In certain preferred embodiments, about 80% or more of the total amount of potassium nitrate has a particle size of about 150 μm or less. In some instances, the oral care composition has about 80% or more, e.g., about 85% or more, about 88% or more, about 91% or more, about 95% or more, about 97% or more, or about 99% or more, of the total amount of potassium nitrate having a particle size of about 150 μm or less, e.g., about 120 μm or less, about 100 μm or less, about 90 μm or less, about 80 μm or less, or about 70 μm or less.

Additionally or alternatively, the oral care compositions may be formulated such that about 99% or more of the total amount of potassium nitrate has a particle size of about 2 mm or less. In some embodiments, about 99% or more of the total amount of potassium nitrate has a particle size of about 1.2 mm or less, about 1 mm or less, about 0.8 mm or less, about 0.6 mm or less, about 0.4 mm or less, about 0.2 mm or less, or about 0.1 mm or less.

The oral care composition may include one or more polyol(s). The polyol(s) may be present in the oral care composition in an amount from about 15 to about 72 wt. %, based on the total weight of the oral care composition. For example, the amount of polyol(s) in the oral care composition may be from about 15 to about 67 wt. %, about 15 to about 57 wt. %, about 15 to about 47 wt. %, about 15 to about 37 wt. %, about 15 to about 32 wt. %, about 15 to about 27 wt. %, about 15 to about 22 wt. %; from about 21 to about 72 wt. %, about 21 to about 62 wt. %, about 21 to about 57 wt. %, about 21 to about 47 wt. %, about 21 to about 42 wt. %, about 21 to about 37 wt. %, about 21 to about 32 wt. %, about 21 to about 27 wt. %; from about 26 to about 72 wt. %, about 26 to about 67 wt. %, about 26 to about 62 wt. %, about 26 to about 57 wt. %, about 26 to about 52 wt. %, about 26 to about 47 wt. %, about 26 to about 42 wt. %, about 26 to about 37 wt. %, about 26 to about 32 wt. %; from about 31 to about 72 wt. %, about 31 to about 67 wt. %, about 31 to about 62 wt. %, about 31 to about 57 wt. %, about 31 to about 52 wt. %, about 31 to about 47 wt. %, about 31 to about 42 wt. %, about 31 to about 37 wt. %; from about 36 to about 72 wt. %, about 36 to about 67 wt. %, about 36 to about 62 wt. %, about 36 to about 57 wt. %, about 36 to about 52 wt. %, about 36 to about 47 wt. %, about 36 to about 42 wt. %; from about 41 to about 72 wt. %, about 41 to about 67 wt. %, about 41 to about 62 wt. %, about 41 to about 57 wt. %, about 41 to about 52 wt. %, about 41 to about 47 wt. %; from about 46 to about 72 wt. %, about 46 to about 67 wt. %, about 46 to about 62 wt. %, about 46 to about 57 wt. %, about 46 to about 52 wt. %; from about 51 to about 72 wt. %, about 51 to about 67 wt. %, about 51 to about 62 wt. %, about 51 to about 57 wt. %; from about 56 to about 72 wt. %, about 56 to about 67 wt. %, about 56 to about 62 wt. %; from about 61 to about 72 wt. %, about 61 to about 67 wt. %, or any range or subrange thereof, based on the total weight of the oral care composition.

The polyol(s) may be chosen from glycols or compounds with numerous hydroxyl groups. The one or more polyols may be liquid at ambient temperature (25° C.). The polyol(s) may be selected from glycerin, glycol, inositol, maltitol, mannitol, sorbitol, xylitol, propylene glycol, polypropylene glycol (PPG), polyethylene glycol (PEG), a block copolymer of PPG and PEG, a saccharide (e.g., fructose, glucose, sucrose and mixtures of saccharides, such as honey), or a combination of two or more thereof. For instance, the oral care composition may comprise maltitol, mannitol, sorbitol, xylitol, a polypropylene glycol (PPG), a polyethylene glycol (PEG), a block copolymer of PPG and PEG, a block copolymer of polyoxypropylene and polyoxyethylene, or a combination or two or more thereof.

The polyol, in certain embodiments, may be selected from propylene glycol, a block copolymer of ethylene oxide and propylene oxide, a polyethylene glycol/polypropylene glycol copolymer, a block copolymer of polyoxypropylene and polyoxyethylene, glycerin, sorbitol, and a combination of two or more thereof. In at least one preferred embodiment, the oral care composition includes polyethylene glycol/polypropylene glycol 116/66 copolymer.

The oral care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof. In some embodiments, the polyol is a polyethylene glycol, such as polyethylene glycol 600 (CAS-25322-68-3). In further embodiments, the oral care compositions comprise one or more polyethylene glycols, e.g., polyethylene glycols in a molecular weight range from 200 to 800. For example, the oral care composition may comprise polyethylene glycol 200, polyethylene glycol 300, polyethylene glycol 400, polyethylene glycol 600, polyethylene glycol 800, or a combination of two or more thereof.

In some cases, the polyol may be a humectant chosen from glycol, sorbitol, glycerin, inositol, maltitol, mannitol, xylitol, propylene glycol, polypropylene glycol (PPG), polyethylene glycol (PEG), a saccharide, polyoxypropylene-polyoxyethylene block copolymer and a combination of two or more thereof.

Additional examples of polyols include those selected from the group consisting of C₂-C₃₂ polyols. The one or more polyols may have from 2 to 32 carbon atoms, from 3 to 16 carbon atoms, or from 3 to 12 carbon atoms. For example, the oral care composition may comprise ethylene glycol, propylene glycol, butylene glycol, hexylene glycol, glycerin, diglycerin, diethylene glycol, and dipropylene glycol, or a combination of two or more thereof. Additional, non-limiting examples of polyols that may, optionally, be incorporated in the oral care composition include alkanediols, such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers, such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof.

An abrasive may be incorporated into the oral care composition. For example, the oral care composition may have from about 1 to about 30 wt. %, based on the total weight of the oral care composition, of one or more abrasive(s). In some embodiments, the abrasive(s) may be present in an amount from about 1 to about 30 wt. %, about 1 to about 27.5 wt. %, about 1 to about 25 wt. %, about 1 to about 22.5 wt. %, about 1 to about 20 wt. %, about 1 to about 18 wt. %, about 1 to about 16 wt. %; about 5 to about 30 wt. %, about 5 to about 27.5 wt. %, about 5 to about 25 wt. %, about 5 to about 22.5 wt. %, about 5 to about 20 wt. %, about 5 to about 18 wt. %, about 5 to about 16 wt. %; about 10 to about 30 wt. %, about 10 to about 27.5 wt. %, about 10 to about 25 wt. %, about 10 to about 22.5 wt. %, about 10 to about 20 wt. %, about 10 to about 18 wt. %, about 10 to about 16 wt. %; about 15 to about 30 wt. %, about 15 to about 27.5 wt. %, about 15 to about 25 wt. %, about 15 to about 22.5 wt. %, about 15 to about 20 wt. %, or about 15 to about 18 wt. %, based on the total weight of the oral care composition.

The abrasives may be chosen from silica (participated silica and/or fused silica), zinc orthophosphate, plastics particles, alumina, hydrated alumina, and calcium pyrophosphate or mixtures thereof. Suitable abrasives that may be included in the oral care composition include, but are not limited to, silica, alumina, insoluble phosphates, calcium carbonate, resinous abrasives, and a combination of two or more thereof. In at least one embodiment, the oral care composition does not include fused silica. In at least one other embodiment, the oral care composition includes fused silica.

The abrasive(s) based on insoluble phosphate salts may be chosen from calcium pyrophosphate, dicalcium orthophosphate dihydrate, tricalcium phosphate, and calcium polymetaphosphate, and a combination of two or more thereof. The total amount of abrasives is preferably comprised primarily of calcium pyrophosphate and, in at least one embodiment, calcium pyrophosphate is the sole abrasive in the oral care composition. For example, the calcium pyrophosphate may comprise about 50 wt. % or more, about 60 wt. % or more, about 70 wt. % or more, about 80 wt. % or more, about 95 wt. % or more, about 98 wt. % or more, or about 99 wt. % or more of the total amount of the abrasive agent in the oral care composition.

Additionally or alternatively, the oral care composition may include one or more an anti-calculus agent, e.g., in an amount from about 0.5 to about 15 wt. %, based on the total weight of the oral care composition. In some embodiments, the oral care composition includes anti-calculus agent(s) in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 15 wt. %, about 5 to about 10 wt. %, about 5 to about 8 wt. %, about 5 to about 6 wt. %; from about 7 to about 15 wt. %, about 7 to about 10 wt. %, about 7 to about 8 wt. %; from about 8 to about 15 wt. %, about 8 to about 12 wt. %, about 8 to about 10 wt. %; from about 10 to about 15 wt. %, about 10 to about 12 wt. %, about 12 to about 15 wt. %, or any range or subrange thereof, based on the total weight of the oral care composition.

The oral care compositions may include anti-calculus agents chosen from phosphates and polyphosphates (e.g., pyrophosphates), polyaminopropanesulfonic acid (AMPS), hexametaphosphate salts, zinc citrate trihydrate, polypeptides, polyolefin phosphates, diphosphonates, and combinations of two or more thereof. Examples of additional anti-calculus agents include tetrasodium pyrophosphate, ammonium tripolyphosphate, hexametaphosphate salt, polycarboxylate polymers, polyvinyl methyl ether/maleic anhydride (PVME/MA) copolymers, or combinations of two or more thereof. In some embodiments, the oral care composition includes one or more alkali metal polyphosphate, such as tetrasodium pyrophosphate. The anti-tarter agents may be a pyrophosphate chosen from dialkali or tetra-alkali metal pyrophosphate salts, such as Na₄P₂O₇ (TSPP), K₄P₂O₇, Na₂K₂P₂O₇, Na₂K₂H₂O₇, and K₂H₂P₂O₇, long chain polyphosphates such as sodium hexametaphosphate, cyclic phosphates such as sodium trimetaphosphate, and a combination of two or more thereof. In at least one embodiment, the anti-calculus agent is selected from tetrasodium pyrophosphate, ammonium tripolyphosphate, hexametaphosphate salt, and a combination thereof.

The oral care compositions may include one or more surfactant(s) in various amounts. The oral care composition may comprise one or more anionic surfactant, one or more cationic surfactant, one or more non-ionic surfactant, or a combination of two or more thereof. For example, the oral care composition may include one or more surfactant(s) in an amount of about 0.5 to about 10 wt. %, based on the total weight of the oral care composition. In some instances, the surfactant(s) is present in the oral care composition in an amount from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The one or more surfactant(s) may comprise an anionic surfactant selected from sulfate based anionic surfactants and non-sulfate based anionic surfactants. For example, the oral care composition may include an anionic surfactant chosen from ammonium lauryl sulfate, ammonium lauryl ether sulfate, sodium dodecyl sulfate, sodium coco-sulfate, ammonium coco-sulfate, and a combination of two or more thereof. In some preferred embodiments, the oral care composition comprises sodium lauryl sulfate, sodium lauryl ether sulfate, or a combination thereof. In further embodiments, one or more anionic surfactant is selected from sodium lauryl sulfate, sodium lauryl ether sulfate, sodium lauryl sarcosinate, sodium cocoyl methyl taurate, sodium monoglyceride sulfate, sodium cetaryl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and a combination of two or more thereof.

Additionally or alternatively, the oral care composition may comprise one or more non-ionic surfactant(s). The one or more non-ionic surfactant(s) may comprise a glucoside, compounds produced by the condensation of alkylene oxide groups (hydrophilic in nature) with an organic hydrophobic compound, which may be aliphatic or alkylaromatic in nature. Examples of glucoside surfactants include decyl glucoside, stearyl glucoside, lauryl glucoside, coco-glucoside, cetearyl glucoside, decyl lauryl glucoside, lauroyl ethyl glucoside, myristoyl ethyl glucoside, oleoyl ethyl glucoside, or a combination of two or more thereof. Further examples of non-ionic surfactants include poloxamers, polyethylene oxide condensates of alkyl phenols, products derived from the condensation of ethylene oxide with the reaction product of propylene oxide and ethylene diamine, ethylene oxide condensates of aliphatic alcohols, acids, and esters, long chain tertiary amine oxides, long chain tertiary phosphine oxides, long chain dialkyl sulfoxides and mixtures of such materials.

The oral care composition may include amphoteric surfactants, such as those chosen from derivatives of aliphatic secondary and tertiary amines, in which the aliphatic radical can be a straight chain or branched and wherein one of the aliphatic substituents contains from about 8 to about 18 carbon atoms and one contains an anionic water-solubilizing group, such as carboxylate, sulfonate, sulfate, phosphate, or phosphonate. Other exemplary amphoteric surfactants are betaines, such as cocamidopropyl betaine, lauryl dimethyl betaine (sold under the trade name Macat LB), cetyl dimethyl betaine, and cocoamphodiacetate. Additional amphoteric surfactants and non-ionic surfactants can be found in U.S. Pat. No. 4,051,234, which is incorporated herein in its entirety for all purposes.

In some embodiments, the oral care composition may include, or in some cases exclude, a fatty amphiphile. The fatty amphiphile may be selected from fatty alcohols, alkoxylated fatty alcohols, fatty phenols, alkoxylated fatty phenols, fatty amides, alkyoxylated fatty amides, fatty amines, fatty alkylamidoalkylamines, fatty alkyoxylated amines, fatty carbamates, fatty amine oxides, fatty acids, alkoxylated fatty acids, fatty diesters, fatty sorbitan esters, fatty sugar esters, methyl glucoside esters, fatty glycol esters, mono, di- and tri-glycerides, polyglycerine fatty esters, alkyl glyceryl ethers, propylene glycol fatty acid esters, cholesterol, ceramides, fatty silicone waxes, fatty glucose amides, phospholipids, and a combination of two or more thereof.

The fatty amphiphile may be present in an amount from about 0.5 to about 10 wt. %, based on the total weight of the oral care composition. For example, the fatty amphiphile may be present in the oral care composition in an amount from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

In some embodiments, the oral care composition has a reduced amount, is substantially free of, or free of fatty amphiphiles. For instance, the oral care composition may comprise about 5 wt. % or less, optionally about 4 wt. % or less, optionally about 3 wt. % or less, optionally about 2 wt. % or less, or optionally about 1 wt. % or less, of fatty amphiphiles. In at least one embodiment, the oral care composition has about 0 wt. % or 0 wt. % of fatty amphiphiles.

The oral care compositions may, in some embodiments, include one or more thickening agent(s). The total amount of thickening agent(s) may be from about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.8 wt. %, about 0.1 to about 0.5 wt. %; from about 0.3 to about 7 wt. %, about 0.3 to about 5 wt. %, about 0.3 to about 4 wt. %, about 0.3 to about 3 wt. %, about 0.3 to about 2.5 wt. %, about 0.3 to about 2 wt. %, about 0.3 to about 1 wt. %, about 0.3 to about 0.8 wt. %; from about 0.5 to about 7 wt. %, about 0.5 to about 5 wt. %, about 0.5 to about 4 wt. %, about 0.5 to about 3 wt. %, about 0.5 to about 2.5 wt. %, about 0.5 to about 2 wt. %, about 0.5 to about 1 wt. %; from about 0.8 to about 7 wt. %, about 0.8 to about 5 wt. %, about 0.8 to about 4 wt. %, about 0.8 to about 3 wt. %, about 0.3 to about 2.5 wt. %, about 0.8 to about 2 wt. %; from about 1 to about 7 wt. %, about 1 to about 5 wt. %, about 1 to about 4 wt. %, about 1 to about 3 wt. %, about 1 to about 2.5 wt. %, about 1 to about 2 wt. %; from about 1.5 to about 7 wt. %, about 1.5 to about 5 wt. %, about 1.5 to about 4 wt. %, about 1.5 to about 3 wt. %, about 1.5 to about 2.5 wt. %; from about 2.5 to about 7 wt. %, about 2.5 to about 5 wt. %, about 2.5 to about 4 wt. %; from about 4 to about 7 wt. %, about 4 to about 5 wt. %; from about 5 to about 20 wt. %, about 5 to about 15 wt. %, about 5 to about 10 wt. %, about 5 to about 7 wt. %; from about 8 to about 20 wt. %, about 8 to about 15 wt. %, about 8 to about 10 wt. %; from about 11 to about 20 wt. %, about 11 to about 15 wt. %, about 8 to about 10 wt. %; from about 14 to about 20 wt. %, about 14 to about 17 wt. %, or any range or subrange thereof, based on the total weight of the oral care composition.

The oral care composition may be formulated to have a reduced amount, be substantially free of, or free of thickening agents. For example, the oral care compositions may have less than about 5 wt. % of thickening agent(s). In some embodiments, the total amount of thickening agent(s) present in the oral care composition is less than about 4 wt. %, less than about 3 wt. %, less than about 2 wt. %, less than about 1 wt. %, less than about 0.5 wt. %, less than about 0.1 wt. %, based on the total weight of the oral care composition. In some instances, the oral care composition may have about 0 wt. % or 0 wt. % of thickening agents, based on the total weight of the oral care composition.

The thickening agent(s) may be selected from an ammonium acryloyldimethyltaurate/VP copolymer, a sodium polyacryloyldimethyltaurate, a polyvinylpyrrolidone, a cross-polymer 6, and a combination of two or more thereof. In at least one preferred embodiment, the thickening agent(s) comprises an ammonium acryloyldimethyltaurate/VP copolymer, a sodium acryloyldimethyltaurate, or a combination of two or more thereof. Illustrative thickening agents include, but are not limited to, polyvinyl pyrrolidone, carbomers (e.g., carboxyvinyl polymers), carrageenans (e.g., Irish moss, carrageenan, iota-carrageenan, etc.), high molecular weight polyethylene glycols (e.g., CARBOWAX.RTM., which is commercially available from The Dow Chemical Company of Midland, Mich.), cellulosic polymers, hydroxyethylcellulose, carboxymethylcellulose, and salts thereof (e.g., CMC sodium), natural gums (e.g., karaya, xanthan, gum arabic, and tragacanth), colloidal magnesium aluminum silicate, and the like, and mixtures or combinations of two or more thereof. In one embodiment, the oral care composition includes a thickening system comprising a polymer selected from polyvinyl pyrrolidone, a polyacrylate, a polymethacrylate, a polyitaconate, an acrylamide, 2-acrylamido-2-methylpropane sulfonic acid (AMPS); and a combination of two or more thereof.

In some embodiments, the oral care compositions may include one or more sweeteners. The oral care composition may include caloric sweeteners and/or non-caloric sweeteners. Examples of non-caloric sweeteners include saccharin, e.g., sodium saccharin, acesulfame, neotame, cyclamate or sucralose; natural high-intensity sweeteners, such as thaumatin, stevioside or glycyrrhizin; or sugar alcohols, such as sorbitol, xylitol, maltitol and mannitol. In at least one embodiment, the oral care composition includes a sweetener selected from sucrose, lactose, maltose, sorbitol, xylitol, sodium cyclamate, perillartine, L-aspartyl-L-phenylalanine methyl ester, saccharine, and a combination of two or more thereof. Examples of caloric sweeteners include sugars, such as fructose, glucose, sucrose, and high fructose syrups.

The one or more sweetener(s) may be present in the oral care composition in an amount from about 0.1 to about 7 wt. %, based on the total weight of the oral care composition. For example, the oral care composition may have a total amount of sweetener(s) from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %; from about 4 to about 7 wt. %, about 4 to about 5 wt. %; from about 5 to about 7 wt. %, or any range or subrange thereof, based on the total weight of the oral care composition.

In some embodiments, the oral care composition has a reduced amount, is substantially free, or is free of caloric sweeteners. For example, the oral care compositions may have about 4 wt. % or less, about 3 wt. % or less, about 2 wt. % or less, about 1 wt. % or less, about 0.5 wt. % or less, or about 0.1 wt. % or less, based on the weight of the oral care composition. In at least one embodiment, the oral care composition contains about 0 wt. % or 0 wt. % of caloric sweeteners, based on the weight of the oral care composition.

The oral care compositions of the present disclosure may include a flavoring agent. The flavoring agent is typically incorporated in the oral care composition at a concentration of about 0.01 to about 3 wt. % by weight of the oral care composition. For example, the amount of flavoring agent(s) present in the oral care composition may be from about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 3 wt. %, about 0.5 to about 2 wt. %, about 0.5 to about 1 wt. %; from about 1 to about 3 wt. %, about 1 to about 2 wt. %; from about 2 to about 3 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

The oral care composition may include a desensitizing agent other than potassium nitrate. Non-limiting examples of desensitizing agent, which may be incorporated into or exclude from the oral care composition, include strontium chloride; potassium nitrate; sodium fluoride; sodium nitrate; acetanilide; phenacetin; acertophan; thiorphan; spiradoline; aspirin; codeine; thebaine; levorphenol; hydromorphone; oxymorphone; phenazocine; fentanyl; buprenorphine; butaphanol; nalbuphine; pentazocine; natural herbs, such as gall nut; Asarum; Cubebin; Galanga; scutellaria; Liangmianzhen; and Baizhi.

The amount of desensitizing agent other than potassium nitrate present in the oral care composition may range from about 0.5 to about 10 wt. %, based on the total weight of the oral care composition. For example, the oral care composition may have desensitizing agent(s) other than potassium nitrate in an amount from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The oral care compositions may include a fluoride ion source. The fluoride ion source may be present in an effective amount. In some cases, the fluoride ion source is present in the oral care composition in an amount of about 0.1 to about 7 wt. %, based on the total weight of the oral care composition. For example, the fluoride ion source may be present in an amount of about 0.1 to about 7 wt. %, about 0.1 to about 5 wt. %, about 0.1 to about 3 wt. %, about 0.1 to about 2 wt. %, about 0.1 to about 1 wt. %; from about 0.4 to about 7 wt. %, about 0.4 to about 5 wt. %, about 0.4 to about 3 wt. %, about 0.4 to about 2 wt. %, about 0.4 to about 1 wt. %; from about 0.8 to about 7 wt. %, about 0.8 to about 5 wt. %, about 0.8 to about 3 wt. %, about 0.8 to about 2 wt. %, about 0.8 to about 1.5 wt. %; from about 1.4 to about 7 wt. %, about 1.4 to about 5 wt. %, about 1.4 to about 3 wt. %; from about 2 to about 7 wt. %, about 2 to about 5 wt. %, about 2 to about 3 wt. %; from about 3 to about 7 wt. %, about 3 to about 5 wt. %, about 3 to about 4 wt. %; from about 4 to about 7 wt. %, about 4 to about 6 wt. %, about 4 to about 5 wt. %; from about 5 to about 7 wt. %, about 5 to about 6 wt. %, or any range or subrange thereof, based on the total weight of the oral care composition.

The fluoride ion source may be selected from soluble fluoride ion salts. For example, the fluoride ion source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides or a combination of two or more thereof. In some embodiments, the fluoride ion source comprises sodium fluoride, sodium monofluorophosphate, or a combination thereof. Additional examples of fluoride ion sources are disclosed in U.S. Pat. Nos. 3,535,421, 4,885,155, and 3,678,154, the disclosures of which are hereby incorporated by reference in their entirety for all purposes.

The oral care compositions may include one or more colorants. Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti. The concentrations of the colorant in the oral care composition may be from trace amount to about 0.6 wt. %, from about 0.1 to about 0.5 wt. %, about 0.2 to about 0.4 wt. %, or about 0.15 to about 0.35 wt. %, based on the total weight of the oral care composition.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges thereof, based on the total weight of the oral care composition.

In another aspect, the present disclosure provides a method of using the oral care compositions disclosed herein. The methods typically comprise applying any of the oral care compositions as described herein to the teeth, e.g., by brushing, rubbing, swishing when the oral care composition is in the form of a dentifrice, or otherwise administering the oral care compositions to the oral cavity of a subject. The oral care compositions can be administered regularly, such as, for example, one or more times per day.

The methods may comprise applying any of the compositions as described herein to the teeth, e.g., by brushing, gargling or rinsing, or otherwise administering the oral care composition to the oral cavity of a subject in need thereof. The oral care composition can be administered regularly, such as, for example, one or more times per day (e.g., twice per day). In various embodiments, administering the compositions of the present disclosure to teeth may provide one or more of the following specific benefits: (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, and/or (xiii) promote systemic health, including cardiovascular health, e.g., by reducing potential for systemic infection via the oral tissues.

### EXAMPLES

### Example 1

An exemplary oral care composition (Ex. A) and a comparative composition (Comp. 1) were prepared in accordance with an aspect of the invention. The formulations for Ex. A and Comp. 1 are shown in Table 1 (below). Ex. A and Comp. 1 both yielded 1000 ppm of fluorine ions. The total amount of hydrogen peroxide in the Cross-linked PVP complexed with Hydrogen Peroxide was about 4 wt. % in Ex. A and Comp. 1.

TABLE 1

| | Ex. A | Comp. 1 |
|---|---|---|
| Ingredient | (wt. %) | (wt. %) |
| Cross-linked PVP complexed with | 1-25 | 1-25 |
| Hydrogen Peroxide | | |
| Potassium Nitrate | 1-10 | — |
| Sodium Monofluorophosphate | 0.1-1 | 0.1-1 |
| Calcium Pyrophosphate | 5-20 | 5-20 |
| Tetrasodium Pyrophosphate | 0.5-2 | 0.5-2 |
| Sodium Acid Pyrophosphate | 0.1-1 | 0.1-1 |
| Sodium Lauryl Sulfate | 0.5-5 | 0.5-5 |
| Polyethylene Glycol / Polypropylene | 1-10 | 1-10 |
| Glycol 116/66 Copolymer | | |
| Polyoxypropylene-Polyoxyethylene | 40-50 | 40-50 |
| Block Copolymer | | |
| Silica | 1-5 | 1-5 |
| Butylated Hydroxytoluene | 0.01-0.1 | 0.01-0.1 |
| Sodium Saccharin and Sucralose | 0.1-1 | 0.1-1 |
| Flavor | 1-5 | 1-5 |
| Total Components | 100 | 100 |

### Example 2

Ex. A was evaluated to determine the stability of potassium (K) in the oral care composition. Hydrogen peroxide, pH, and the amount of soluble fluoride were also measured to ensure that the potassium nitrate did not impact the performance of Ex. A. The results of the evaluation of Ex. A are shown in Table 2 (below).

TABLE 2

| | Active | Potassium | pH (10% | Soluble | |
|---|---|---|---|---|---|
| | Oxygen | (K) | Solution) | Fluoride | |
| Specification | 3.6-4.4% | 1.74-2.14% | 7.5-8.5 | 850-1150 | ppm |
| (initial) | | | | | |
| Specification | ≥2.8% | 1.70-2.20% | 6.2-8.5 | ≥600 | ppm |
| (aged) | | | | | |
| Initial 30 C./65% RH | 4.1% | 2.03% | 7.9 | 1080 | ppm |
| 1 MO 40 C./75% RH | 4.1% | 1.92% | 7.7 | 1120 | ppm |
| 2 MO 40 C./75% RH | 4.0% | 1.70% | 7.7 | 1100 | ppm |
| 3 MO 40 C./75% RH | 3.9% | 1.92% | 7.8 | 1130 | ppm |
| 3 MO 30 C./65% RH | 4.0% | 1.80% | 7.8 | 1090 | ppm |

### Example 3

A randomized, double-blind, single-center and two-cell parallel-group clinical study was designed to assess the anti-sensitivity efficacy of a non-aqueous toothpaste containing 5 wt. % of KNO₃ and 4 wt. % of HP as compared to a matching placebo toothpaste (which did not include the potassium nitrate) on dentin hypersensitivity reduction after 8 weeks of product use.

Specifically, dentin hypersensitivity was assessed via tactile stimuli and air blast stimuli. The volunteers were assigned to two groups of 38 volunteers, with one group being instructed to brush their teeth with Ex. A and the second group being instructed to brush their teeth with Comp. 1. The volunteers were instructed to brush their teeth once in the morning and once in the evening for a period of 2 minutes for 8 weeks. A summary of the age and gender for the volunteers is shown in Table 3.

TABLE 3

| | Number of Volunteers | | | Age of Volunteers | |
|---|---|---|---|---|---|
| Treatment | Male | Female | Total | Mean (S.D.) | Range |
| Test Group | 12 | 26 | 38 | 45.11 | 19.58-70 |
| | | | | (13.77) | |
| Placebo | 18 | 20 | 38 | 46.6 | 20.75-67.75 |
| Group | | | | (11.61) | |
| All Treatment | 30 | 46 | 76 | 45.86 | 19.58-70 |
| Groups | | | | (12.67) | |

The baseline-adjusted tactile hypersensitivity scores and 95% confidence intervals measured after 4 weeks. A negative percentage indicates no improvement in dentin hypersensitivity. The mean baseline scores was 10.92 for the volunteers assigned to the group using Ex. A and 11.84 for the volunteers assigned to the group using Comp. 1. No statistically significant difference was indicated between the treatment groups with respect to either tactile (p=0.230) or air blast (p=0.762) hypersensitivity scores at baseline. A summary of the mean tactile at the baseline examination for the volunteers is shown in Table 4, which is provided below.

TABLE 4

| | | Number of | Base Line | 4-Week | 8-Week |
|---|---|---|---|---|---|
| | Treatment | Volunteers | Mean (S.D.) | Mean (S.D.) | Mean (S.D.) |
| Tactile | Ex. A Group | 38 | 10.92 (2.81) | 18.03 (7.22) | 21.84 (6.82) |
| Hypersensitivity | | | | | |
| | Comp. 1 | 38 | 11.84 (3.75) | 11.71 (3.54) | 11.97 (3.77) |
| | Group | | | | |

For the group using Ex. A, the baseline-adjusted mean [95% CI] tactile hypersensitivity scores recorded after 4 weeks of product use were 18.39 [20.56, 23.82]. For the group using Comp. 1, the baseline-adjusted mean [95% CI] tactile hypersensitivity scores recorded after 4 weeks of product use were 11.34 [9.99, 13.25].

The percent changes from the baseline score was 65.1% for the group using Ex. A and −1.1% for the group using Comp. 1. The improvement from the baseline score for the group using Ex. A was a statistically significant (p<0.001), whereas Comp. 1 did not achieve a statistically significant change from the baseline score (p=0.324). Ex. A exhibited an improvement of 62.2% in tactile hypersensitivity after 4 weeks of product relative to the Comp. 1. Table 5 presents a summary of the baseline-adjusted tactile hypersensitivity scores and 95% confidence intervals measured after 4 weeks of the Experiment.

TABLE 5

| | | | Within Treatment | | Between- Treatment | |
|---|---|---|---|---|---|---|
| | | | Analysis | | Comparison | |
| | Adj. 4-Week | Adj. 4-Week | Percent | | Percent | |
| Treatment | Mean (S.E.) | 95% CI | Change | Sig. | Difference | Sig. |
| Ex. A | 18.39 (0.83) | 20.56, 23.82 | 65.1% | p < 0.001 | 62.2% | p < 0.001 |
| Comp. 1 | 11.34 (0.83) | 9.99, 13.25 | −1.1% | p = 0.324 | | |

Tactile hypersensitivity scores were determined for the two groups after 8 weeks of product use. Specifically, the baseline-adjusted mean [95% CI] tactile hypersensitivity score after 8 weeks for the group using Example Composition A was determined to be 22.19 [16.72, 20.06]. For the group using Comp. 1, the baseline-adjusted mean [95% CI] tactile hypersensitivity scores after 8 weeks of use was 11.62 [9.67, 13.01].

The percent improvement of the baseline adjusted mean score achieved by Ex. A 100.0%, while the percent improvement of the baseline adjusted mean score of Comp. 1 was 1.1%. The percent improvement from baseline observed for the Test Group was statistically significant (p<0.001). Additionally, Ex. A exhibited an a statistically significant (p<0.001) improvement of 91.0%, relative to Comp. 1, in tactile hypersensitivity scores after 8 weeks of product use. A summary of the tactile hypersensitivity scores at the 8-Week of the evaluation is provided in Table 6.

TABLE 6

| | | | Within Treatment | | Between- Treatment | |
|---|---|---|---|---|---|---|
| | | | Analysis | | Comparison | |
| | Adj. 8-Week | Adj. 8-Week | Percent | | Percent | |
| Treatment | Mean (S.E.) | 95% CI | Change | Sig. | Difference | Sig. |
| Ex. A | 22.19 (0.8) | 16.72, 20.06 | 100% | p < 0.001 | 91% | p < 0.001 |
| Comp. 1 | 11.62 (0.8) | 9.67, 13.01 | 1.1% | p = 0.571 | | |

The volunteers were also evaluated for dentine sensitivity using air blast hypersensitivity evaluation. For air blast sensitivity, the mean baseline scores were 2.79 for the volunteers assigned to the group using Ex. A and 2.82 for the volunteers assigned to the group using the Comp. 1. A summary of the mean tactile and air blast scores measured at the baseline examination for the volunteers is shown in Table 7, which is provided below.

TABLE 7

| | | Number of | Base Line | 4-Week | 8-Week |
|---|---|---|---|---|---|
| | Treatment | Volunteers | Mean (S.D.) | Mean (S.D.) | Mean (S.D.) |
| Air Blast | Ex. A Group | 38 | 2.79 (0.38) | 2.01 (0.69) | 1.66 (0.66) |
| Hypersensitivity | | | | | |
| | Comp. 1 | 38 | 2.82 (0.38) | 2.84 (0.35) | 2.78 (0.4) |
| | Group | | | | |

The baseline-adjusted mean [95% CI] air blast hypersensitivity scores after 4 weeks for the group using Ex. A was 2.02 [1.49, 1.83]. For the group using Comp. 1, the baseline-adjusted mean [95% CI] air blast hypersensitivity scores after 4 weeks of use was 2.83 [2.60, 2.94].

The percent change from the baseline score for the group using Ex. A was 27.9%, whereas the percentage change from the baseline score for the group using Comp. 1 was-0.7%. The improvement achieved by Ex. A was determined to be statistically significant (p<0.001), while the percent change from baseline observed for group using Comp. 1 was not statistically significant (p=0.160). Ex. A exhibited a statistically significant (p<0.001) reduction of 28.6% in air blast hypersensitivity scores after 4 weeks of product use relative to the Comp. 1. A summary of the baseline-adjusted air blast hypersensitivity scores and 95% confidence intervals measured after 4 weeks is shown in Table 8.

TABLE 8

| | | | Within Treatment | | Between- Treatment | |
|---|---|---|---|---|---|---|
| | | | Analysis | | Comparison | |
| | Adj. 4-Week | Adj. 4-Week | Percent | | Percent | |
| Treatment | Mean (S.E.) | 95% CI | Change | Sig. | Difference | Sig. |
| Ex. A | 2.02 (0.08) | 1.49, 1.83 | 27.9% | p < 0.001 | 28.6% | p < 0.001 |
| Comp. 1 | 2.83 (0.08) | 2.60, 2.94 | −0.7% | p = 0.16 | | |

Air blast hypersensitivity scores were determined for the two groups after 8 weeks of product use. Specifically, the baseline-adjusted mean [95% CI] air blast hypersensitivity score after 8 weeks for the group using Ex. A was determined to be was 1.66 [1.85, 2.19]. For the group using Comp. 1, the baseline-adjusted mean [95% CI] air blast hypersensitivity scores after 8 weeks of use was 2.77 [2.66, 3.00].

The percentage reductions from baseline-adjusted mean was 40.5% for the group using Ex. A and 1.4% for the group using Comp. 1. The percent reduction from baseline-adjusted mean observed for the group using Ex. A was statistically significant (p<0.001), whereas the percent reduction from baseline observed for the group using Comp. 1 was not statistically significant (p=0.373). Ex. A achieved a exhibited a statistically significant (p<0.001) reduction of 40.1% in air blast hypersensitivity scores, relative to Comp. 1, after 8 weeks of product use. A summary of the results of the air blast hypersensitivity scores after 8 weeks is shown in Table 9.

TABLE 9

| | | | Within Treatment | | Between- Treatment | |
|---|---|---|---|---|---|---|
| | | | Analysis | | Comparison | |
| | Adj. 8-Week | Adj. 8-Week | Percent | | Percent | |
| Treatment | Mean (S.E.) | 95% CI | Change | Sig. | Difference | Sig. |
| Ex. A | 1.66 (0.09) | 1.85, 2.19 | 40.5% | p < 0.001 | 40.1% | p < 0.001 |
| Comp. 1 | 2.77 (0.09) | 2.66, 3.0 | 1.4% | p = 0.373 | | |

The results of this randomized and double-blind clinical study support the conclusion that Ex. A provides a statistically significant improvement in dentin hypersensitivity after 4 and 8 weeks of product use as compared to a matching placebo, namely Comparative Composition 1. For Tactile Hypersensitivity, Ex. A achieved statistically significant improvements in dentin tactile hypersensitivity after 4 and 8 weeks of product use (62.2% and 90.1%, respectively) relative to Comp. 1. For air blast hypersensitivity, Ex. A achieved statistically significant improvements in dentin air blast hypersensitivity after 4 and 8 weeks of product use (28.6% and 40.1%, respectively) relative to Comp. 1. Accordingly, Ex. A is clinically proven to provide anti-hypersensitivity benefit after 8-week usage despite the limited solubility of KNO₃ in non-aqueous formula, which was surprising.

### Example 4

A consumer study of Ex. A and Comp. 1 was conducted. The participants in the consumer study were asked to brush their teeth with Ex. A or Comp. 1, at least twice per day, as they normally would use a dentifrice over the course of 14 days. After 14 days, participants completed a questionnaire.

The consumer study results, which are based on the questionnaire, showed that Ex. A achieved an improved consumer acceptance in multiple key features relative to Comp. 1. Table 10 presents a summary of the consumers' preference of Ex. A and Comp. 1 relative to dentifrice products on the market. The category of “Definitely would buy” and “Def. or Prob. would buy” were 47 and 76 for the test sample containing 5 wt. % of KNO₃, which is statistically significant from 36 and 64 from the matching placebo. Table 10 is provided below.

TABLE 10

| | User Ranking | Comp. 1 | Example A |
|---|---|---|---|
| Financial | Definitely Would Buy | 36 | 47 |
| Key | | (Below | (Above |
| Measures | | Average) | Average) |
| | Probably Would Buy | 28 | 28 |
| | Definitely or Probably | 64 | 76 |
| | Would Buy | | |
| | Might or Might Not Buy | 17 | 10 |
| | Probably Would Not Buy | 12 | 11 |
| | Definitely Would No buy | 8 | 3 |
| | Trial Units Among Favs | 1.5 | 1.5 |
| | Frequency Among Favs | 11.8 | 12.8 |

Table 11 presents the factor metrics scores for subjects who completed the 2-Week consumer study. In the product delivery category, Ex. A achieved 60 for “Better than expected”, which is statistically different compared to 44 from Comp. 1. In the product loyalty category, Ex. A received higher scores in both metrics, which were 3.6 in “Uniqueness” and 3.4 in “Value”, compared with the 3.4 in “Uniqueness” and 3.1 in “Value” for Comp. 1. Table 11 is provided below.

TABLE 11

| | | |
|---|---|---|
| | Comp. 1 | Example A |
| | Post-Use: | Post-Use: |
| User Ranking | N = 225 | N = 123 |

| | |
|---|---|
| Factors | Product Delivery |

| | | | |
|---|---|---|---|
| for | Better than Expected | 44 | 60 |
| Success | Same as Expected | 34 | 29 |
| | Worse than Expected | 21 | 11 |

Product Loyalty

| | | |
|---|---|---|
| Uniqueness | 3.4 | 3.6 |
| Value | 3.1 | 3.4 |

Table 12 presents a summary of Product Diagnostic Attributes (Top Box) scores for subjects who completed the 2-Week consumer study. Ex. A obtained higher scores in all categories, except one category—“has a pleasant texture/consistency”—for which Ex. A showed an equal result as compared to Comp. 1. Although the only difference of Ex. A and Comp. 1 is the presence of potassium nitrate, it is believed that adding the potassium nitrate in an amount of 5 wt. % provides other benefits in addition to the clinically proven anti-hypersensitivity benefit, such as a higher consumer perception for “is safe for my teeth”, “freshens my breath”, “makes me feel confident”, “whitens teeth effectively”, “has a taste I like”, “helps manage sensitivity”, “effectively removes stains”, “whitens teeth quickly”, and “prevents future stains”. Thus, Ex. A surprisingly is believed to achieve an improvement in the consumers' acceptance on product safety, flavor delivery and whitening efficacy. Table 12 is provided below.

TABLE 12

| | User Agreement | Comp. 1 | Example A |
|---|---|---|---|
| Post-Use | Is safe for my teeth | 37% | 48% |
| Diagnostic | Freshens my breath | 35% | 47% |
| Attributes | Makes me feel confident | 31% | 38% |
| (ATT) | Has a pleasant texture/consistency | 28% | 28% |
| Top | Whitens teeth effectively | 25% | 33% |
| Box | Has a taste I like | 24% | 35% |
| | Helps manage sensitivity | 24% | 41% |
| | Effectively removes stains | 22% | 32% |
| | Whitens teeth quickly | 21% | 33% |
| | Prevents future stains | 20% | 27% |

What is claimed is:

1. An oral care composition comprising:
a whitening agent;
potassium nitrate, wherein about 80% or more of the total amount of potassium nitrate has a particle size of about 500 μm or less; and
water in an amount of about 8 wt. % or less, wherein all weight percentages are based on the total weight of the oral care composition.

2. The oral care composition according to claim 1, wherein the oral care composition is substantially free, or free, of water.

3. The oral care composition according to claim 1, wherein about 99% or more of the total amount of potassium nitrate has a particle size of about 2 mm or less.

4. The oral care composition according to claim 3, wherein about 99% or more of the total amount of potassium nitrate has a particle size of about 1.2 mm or less.

5. The oral care composition according to claim 1, wherein about 80% or more of the total amount of potassium nitrate has a particle size of about 150 μm or less.

6. The oral care composition according to claim 1, wherein the whitening agent is selected from hydrogen peroxide, cross-linked polyvinylpyrrolidone complexed with hydrogen peroxide, vinylpyrrolidone copolymer complexed with hydrogen peroxide, a silica hydrogen peroxide complex, and a combination of two or more thereof.

7. The oral care composition according to claim 1, wherein the whitening agent comprises polyvinylpyrrolidone complexed with hydrogen peroxide.

8. The oral care composition according to claim 7, wherein the whitening agent further comprises hydrogen peroxide.

9. The oral care composition according to claim 1, wherein the whitening agent is present in an amount from about 1 to about 40 wt. %.

10. The oral care composition according to claim 1, wherein the total amount of hydrogen peroxide, inclusive of complexed and/or non-complexed hydrogen peroxide, present in the oral care composition is from about 1 to about 8 wt. %.

11. The oral care composition according to claim 1 further comprising a polyol in amount for from about 15 to about 72 wt. %, wherein the polyol is selected from propylene glycol, a block copolymer of ethylene oxide and propylene oxide, a polyethylene glycol/polypropylene glycol copolymer, glycerin, sorbitol, and a combination of two or more thereof.

12. The oral care composition according to claim 1 further comprising a fatty amphiphilic selected from fatty alcohols, alkoxylated fatty alcohols, fatty phenols, alkoxylated fatty phenols, fatty amides, alkyoxylated fatty amides, fatty amines, fatty alkylamidoalkylamines, fatty alkyoxylated amines, fatty carbamates, fatty amine oxides, fatty acids, alkoxylated fatty acids, fatty diesters, fatty sorbitan esters, fatty sugar esters, methyl glucoside esters, fatty glycol esters, mono, di- and tri-glycerides, polyglycerine fatty esters, alkyl glyceryl ethers, propylene glycol fatty acid esters, cholesterol, ceramides, fatty silicone waxes, fatty glucose amides, phospholipids, and a combination of two or more thereof.

13. The oral care composition according to claim 1 further comprising an abrasive selected from alumina, insoluble phosphates, calcium carbonate, resinous abrasives, silica, and a combination of two or more thereof.

14. The oral care composition according to claim 1 further comprising an anti-calculus agent in an amount from about 0.5 to about 15 wt. % wherein the anti-calculus agent is selected from selected from tetrasodium pyrophosphate, ammonium tripolyphosphate, hexametaphosphate salt, and a combination thereof.

15. The oral care composition according to claim 1 further comprising a sweetener selected from sucrose, lactose, maltose, sorbitol, xylitol, sodium cyclamate, perillartine, L-aspartyl-L-phenylalanine methyl ester, saccharine, and a combination of two or more thereof.

16. The oral care composition according to claim 1 further comprising a fluoride source selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

17. The oral care composition according to claim 1 further comprising an anionic surfactant selected from ammonium lauryl sulfate, ammonium lauryl ether sulfate, sodium dodecyl sulfate, sodium coco-sulfate, ammonium coco-sulfate, and a combination of two or more thereof. In some preferred embodiments, the oral care composition comprises sodium lauryl sulfate, sodium lauryl ether sulfate, and a combination of thereof.

18. An oral care composition comprising:
a whitening agent;
potassium nitrate, wherein about 80% or more of the total amount of potassium nitrate has a particle size of about 150 μm or less;
from about 15 to about 72 wt. % of a polyol, wherein the polyol consists of a glycol, a polypropylene glycol (PPG), a polyethylene glycol (PEG), a block copolymer of PPG and PEG a block copolymer of ethylene oxide and propylene oxide, glycerin, mannitol, maltitol, xylitol, inositol, sorbitol, a block copolymer of polyoxypropylene and polyoxyethylene and a combination of two or more thereof; and
about 3 wt. % or less of water, wherein all weight percentages are based on the total weight of the oral care composition.

19. The oral care composition of claim 18, wherein the oral care composition provides a reduction in tactile hypersensitivity of about 84% or more after 8 weeks of using the oral care composition at least twice a day.

20. The oral care composition of claim 18, wherein the oral care composition provides a reduction in air blast hypersensitivity of about 41% or more after 8 weeks of using the oral care composition at least twice a day.
